(19) United States
(12) Patent Application Publication
(10) Pub. No.: US 2017/0196572 A1
(43) Pub. Date: Jul. 13, 2017

(54) ELASTOMERIC RESILIENT HANDLE SLEEVE

(71) Applicant: Farrell E. Robinson, Vestavia, AL (US)

(72) Inventors: Farrell E. Robinson, Vestavia, AL (US); Lucus Parker Kohn, Moody, AL (US); Shelby Rice, Birmingham, AL (US); Dillon Ezekiel Smith, McCalla, AL (US); Alex F. Farris, III, Birmingham, AL (US)

(21) Appl. No.: 15/335,372

(22) Filed: Oct. 26, 2016

Related U.S. Application Data
(60) Provisional application No. 62/276,697, filed on Jan. 8, 2016.

(51) Int. Cl. A61B 17/16

(52) U.S. Cl. CPC A61B 17/1633; A61B 17/1606; A61B 2017/0042

(57) ABSTRACT

An elastomeric sleeve which is a hollow tube having a first side, a second opposite side, a top end, and a bottom end, wherein the top end is open and the bottom end is closed. The second opposite side has a slit extending from the top end towards the bottom end. The slit and the open top end make the sleeve easy to place over the handle and to remove it from the handle. The closed bottom end facilitate the sleeves remaining on the handle during use. The slit may have an opening so that handle elements, such as leaf springs, may extend therethrough. The sleeve may have a cover for a horn member on a handle of a rongeur. The cover may have a flange at a base of the cover, thereby forming a cushion. The cover and cushion reduce or eliminate fatigue and trauma to the thenar muscles and surrounding nerves. The sleeve may also have a thumb support.

### CROSS-REFERENCE TO RELATED APPLICATIONS

This application claims priority of U.S. Provisional Application Ser. No. 62/276,697 filed Jan. 8, 2016, the disclosure of which is incorporated herein by reference.

### FIELD OF THE INVENTION

This invention relates to elastomeric resilient sleeves for handles and, particularly, for handles of surgical instruments such as a rongeur.

### BACKGROUND OF THE INVENTION

Many types of surgical instruments have handles so that a surgeon can apply force through the instrument to produce a desired surgical result. These kinds of surgical instruments can result in repetitive motion injuries to the hands and wrists of surgeons due to the repetitive movements involved in applying force to the handles. Conventional surgical instruments have handles that provide inadequate tactile control for certain surgical procedures. In addition, the smooth surfaces of conventional handles require surgeons to expend energy squeezing the handle with his or her palm and fingers to maintain adequate control of the surgical instrument while making incremental surgical movements.

The surgeon's need for comfort and precision has led to an increased focus on the ergonomics of surgical instruments. Hand size has been shown to be an important variable when designing surgical hand tools (Surg Endosc. 2004 Mar;18(3):508-12). Women surgeons experience more discomfort in their hands than their male counterparts (Surg Endosc. 2014 Apr;28(4): 1051-5).

Comfort grips are used in craftsman hand tools and in sports equipment. However, these grips are permanent and are usually composed of polymers that are relatively rigid. They are designed with grooves and ridges to improve maneuverability and grip force. These types of grips are not suitable for surgical instruments because they do not provide adequate tactile control for surgical manipulations and are vulnerable to inadequate cleaning and contamination, leading to surgical site infection.

An ergonomic surgical scalpel sleeve is known for use with microsurgical instruments (U.S. Pat. No. 5,578,050). This sleeve is a straight hollow tube open at both ends and is made of silicone or thermoplastic material. This sleeve is useful only for instruments manipulated with the thumb and forefinger and would not be useful for an instrument such as a bone rongeur. FIG. 1 illustrates a side view of a typical bone rongeur 10 used for bone dissection. The rongeur 10 has a front handle 11 and a rear handle 12, shown with leaf springs 13 which bias the handles 11 and 12 in an open position 14. The rongeur 10 has a straight shank portion 15 with a bite end member 16. The rongeur 10 has a movable shank member 17, shown by arrows 18, which moves relative to stationary shank member 19. The movable shank member 17 opens and closes the bite end member 16 for dissecting bone by means of the surgeon pulling the front handle 11 to the rear handle 12 and then allowing the handles 11 and 12 to spread apart by the force of the leaf springs 13. A horn member 20 is positioned near the top end of rear handle 12. The surgeon's hand engages the horn member 20 at the junction of the thumb and the palm of the hand. The horn member 20 prevents the surgeon's hand from slipping upward along the rear handle 12 as the surgeon operates the rongeur 10. It can be seen from FIG. 1 that the handles 11 and 12 are curved to facilitate the surgeon's grip of the handles. A surgeon may operate the rongeur for up to four hours which can produce considerable discomfort and pain in the hand, especially at the junction of the thumb and the palm of the hand. In some cases chronic irritation and inflammation can occur. There exists a need for surgical instrument handle sleeves which increase tactile control and reduce repetitive motion injuries and cumulative trauma, while reducing the amount of pressure required to be exerted by the surgeon's hand to maintain control of the surgical instrument.

### SUMMARY OF THE INVENTION

The elastomeric sleeve for a handle is formed of a hollow tube having a first side, a second opposite side, a top end, and a bottom end, wherein the top end is open the the bottom end is closed. The second opposite side has a slit extending from the top end towards the bottom end. The slit may have an opening to accommodate structural features that extend outward from the handle. The sleeve is made, preferably, of compressible material. The sleeve may have a cover for a horn member on a handle. The cover may have a flange at a base of the cover, wherein the flange forms a cushion for engaging the upper thenar eminence. The sleeve may also have a thumb support, wherein the thumb support, preferably, extends from the base of the cover.

An advantage of the elastomeric handle sleeve of the present invention is that the sleeve can be easily slipped on and off the surgical handle and can be disposed of after use.

Another advantage is a handle sleeve that is remarkably comfortable.

Another advantage is a handle sleeve that reduces hand fatigue from operating the handles of a surgical instrument.

Another advantage is a handle sleeve that increases the surgeon's accuracy in the manipulation of the surgical instrument.

Another advantage is a cover on the handle sleeve for the horn member of a rongeur, wherein the cover reduces or eliminates fatigue and trauma to the thenar muscles and surrounding nerves, thereby eliminating repetitive motion injuries resulting from operating the rongeur.

Another advantage is handle sleeve that fully engages the space of the thenar eminence, especially at the junction of the thumb and forefinger so that the user can more effectively keep a ronguer remarkably steady during surgical use.

### BRIEF DESCRIPTION OF THE DRAWINGS

FIG. 1 illustrates a side view of a typical prior art bone rongeur used for bone dissection.

FIG. 2 illustrates a rear side perspective view of the handle sleeves positioned on the front and rear handles of a rongeur.

FIG. 3 shows a view of a first side of a front handle sleeve.

FIG. 4 shows a view of a second opposite side of the front handle sleeve.

FIG. 5 shows a side view of the front handle sleeve.

FIG. 6 shows a view of a first side of rear handle sleeve.

FIG. 7 shows a view of a second opposite side of the rear handle sleeve.

FIG. 8 shows a side view of the rear handle sleeve.

FIG. 9 shows a longitudinal sectional view of an alternate embodiment of a horn member cover of the rear handle sleeve.

FIG. 10 shows a cross sectional view through the horn member cover, illustrating the position of a cushion at the base of the horn member cover.

FIG. 11 shows an illustration of hand from the palmer surface aspect.

FIG. 12 shows a top perspective view of an alternant embodiment of the rear sleeve.

FIG. 13 shows a side perspective view of the rear sleeve of FIG. 12 positioned in the palmer surface of a hand.

FIG. 14 shows another side perspective view of the rear sleeve of FIG. 12 positioned in the palmer surface of a hand.

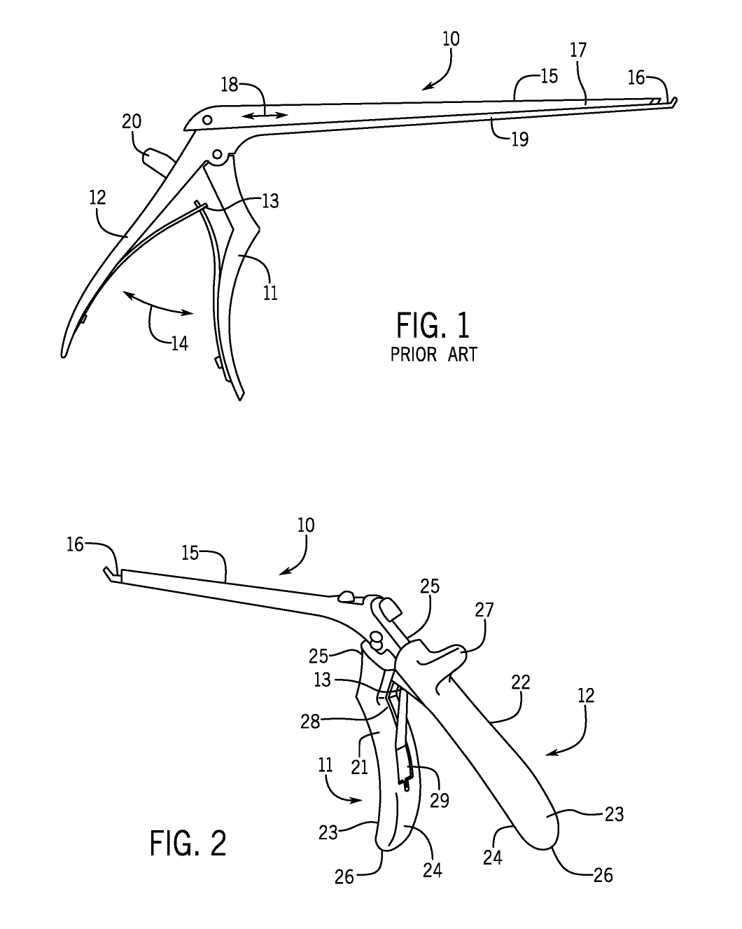

### DETAILED DESCRIPTION OF THE INVENTION

While the following description details the preferred embodiments of the present invention, it is to be understood that the invention is not limited in its application to the details of the method described herein, since the invention is capable of other embodiments and of being practiced in various ways.

The invention is an elastomeric resilient sleeve in the shape of a hollow tube that is contoured to the shape of a surgical instrument handle. The sleeve increases tactile control of the surgical instrument while reducing repetitive motion injuries and cumulative trauma. The sleeve also reduces the amount of pressure required to be exerted by the surgeon's hand to maintain control of the surgical instrument. The sleeve can also be sterilized and reused.

FIG. 2 illustrates a rear side perspective view of the handle sleeves 21 and 22 positioned on the front 11 and rear 12 handles of a rongeur 10. Each sleeve has a first side 23, a second opposite side 24, a top end 25, and a bottom end 26. Rear handle 12 has a horn member cover 27 which extends out from the first side 23 near the top end 25. The top ends 25 are open and the bottom ends 26 are closed. The opposite sides 24 have a slit 28 which extends from the open tops 25 to about one third to two thirds down the longitudinal axes of the sleeves 21 and 22. With instruments that have devices such as leaf springs 13 there can be an opening 29 on the opposite sides 24 so that the leaf springs 13 can extend therethrough. The longitudinal slits 28 and the open tops 25 make the sleeves 21 and 22 easy to place over the handles 11 and 12 of the rongeur 10 and also easy to remove them from the handles 11 and 12 after use. The closed bottom ends 26 facilitate the sleeves 21 and 22 remaining on the handles 11 and 12 during use by the surgeon.

The thickness of the walls of the sleeves 21 and 22 is, preferably, 6-25 mm to provide a comfortable grip and to assist in the application of manual force to the handle or handles of a surgical instrument. Sleeves having a wall thickness of 3-5 mm may also be used. The walls of the sleeve are compressible, preferably, to 30% to 60% of resting wall thickness. This characteristic of the elastomeric material allows the elastomeric material to conform to the shape of the fingers, palm, and thumb just sufficiently to distribute manual force evenly over the fingers, palm, and thumb. As a result, force generated in operating a surgical handle or handles is not directed unacceptably more to any one area of the palm, fingers, or thumb. The elastomeric polymer may be formulated to withstand sterilization up to 300 degrees centigrade. The elastomeric resilient handle sleeve is made of a liquid molding polymer, preferably SterAlloy FDG (Hapco, Inc., Hanover, Mass. Innovative polymers, Inc., Saint John, Mich.) having a durometer ranging from 20 to 70.

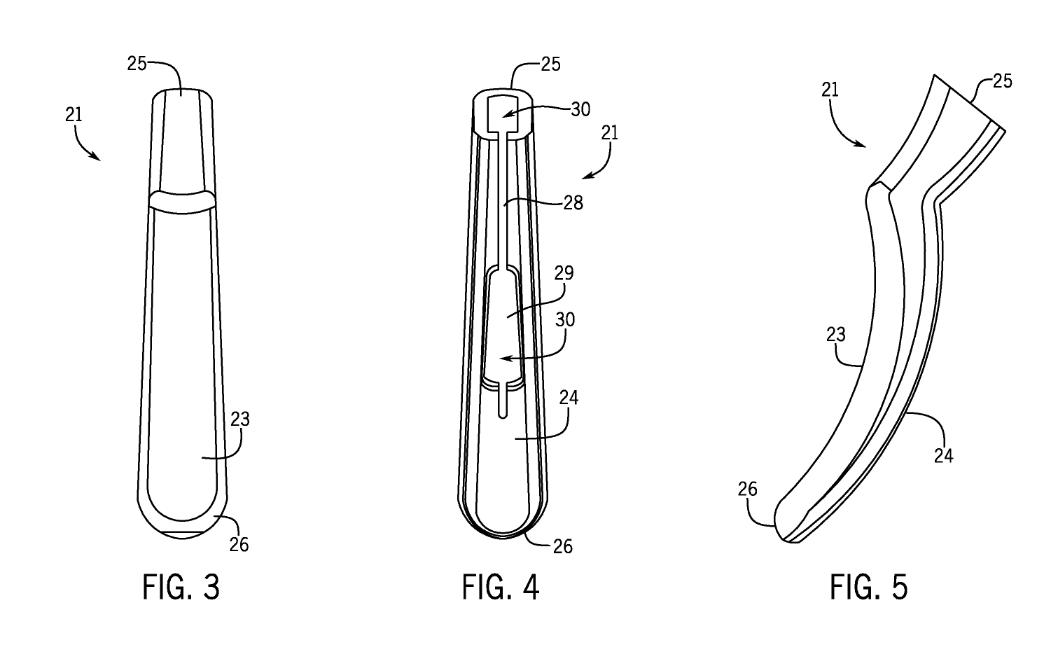

FIG. 3 shows a view of the first side 23 of the front sleeve 21. FIG. 4 shows a view of the second opposite side 24 of the front sleeve 21, further illustrating the open top end 25, the slit 28, the opening 29 along the slit 28, and the hollow interior 30 of the sleeve 21. FIG. 5 shows a side view of the front sleeve 21.

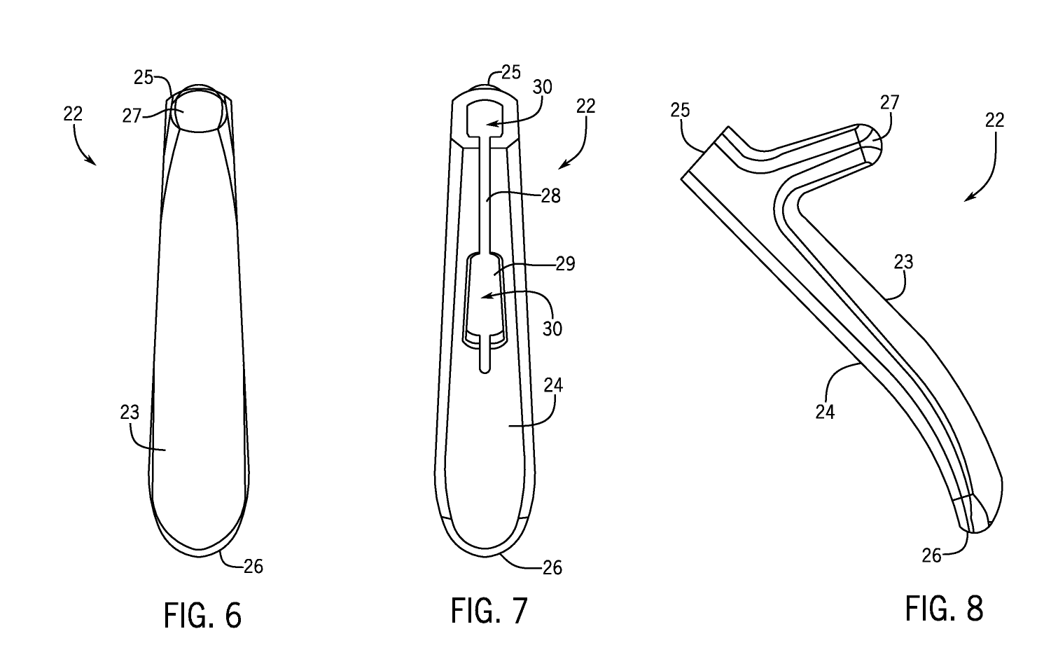

FIG. 6 shows a view of the first side 23 of the rear sleeve 22 illustrating the position of a horn member cover 27. FIG. 7 shows a view of the second opposite side 24 of the rear sleeve 22, further illustrating the open top end 25, horn member cover 27, the slit 28, the opening 29 along the slit 28, and the hollow interior 30 of the sleeve 21. FIG. 8 shows a side view of the rear sleeve 22.

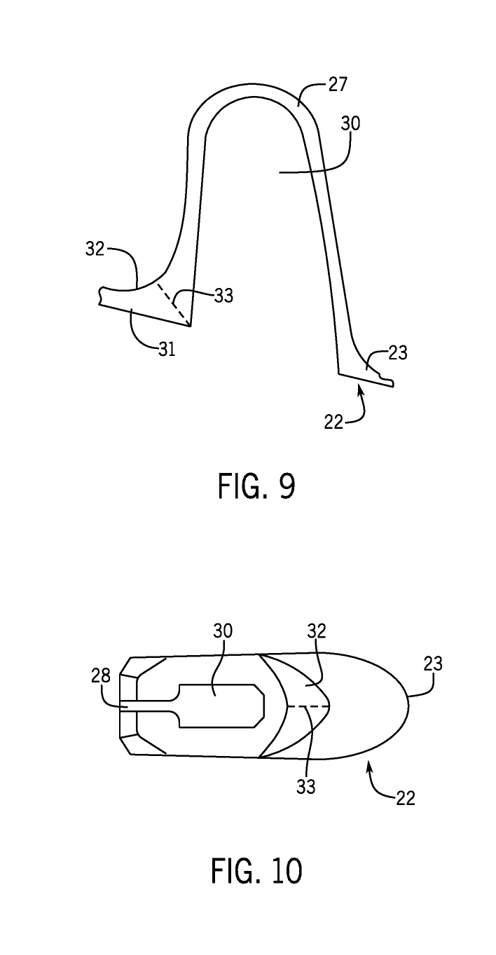

FIG. 9 shows a longitudinal sectional view of an alternate embodiment of the horn member cover 27 of the rear sleeve 22. The horn member cover 27 has a flange 31 which extends around the base of the horn member cover 27 and extends up from the sleeve 22 to form a cushion 32. FIG. 10 shows a cross sectional view through the horn member cover 27 illustrating the position of the cushion 32 at the base of the horn member cover 27. The cushion 32 has a thickness, shown by the dashed line 33, of 3 mm to 10 mm, preferably 5 mm and is made of the same elastomeric material as the rest of the sleeve 22.

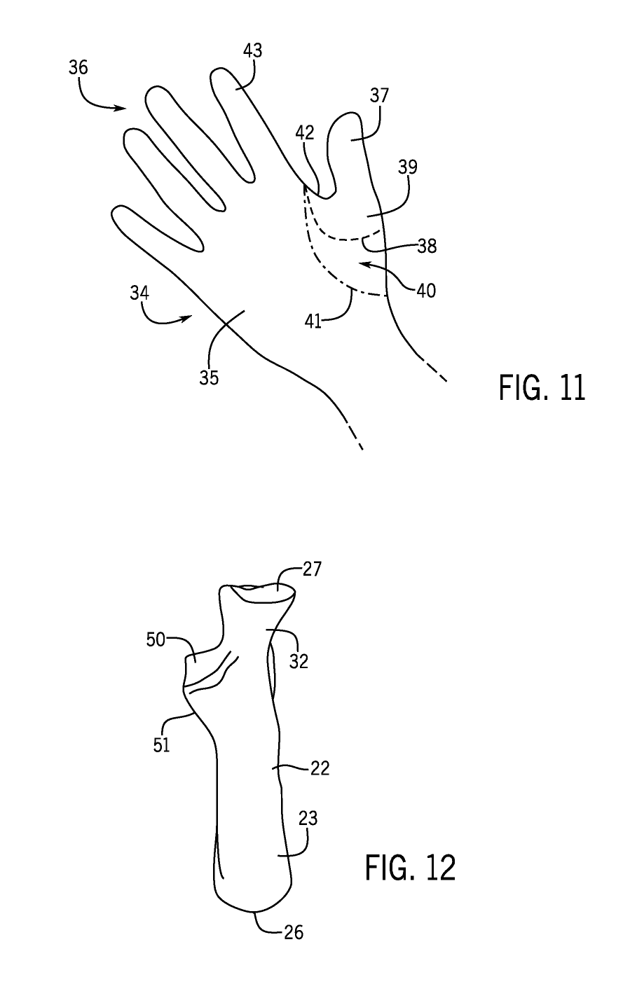

FIG. 11 shows an illustration of hand 34 from the palmer surface 35 aspect. Also shown are the fingers 36 and the thumb 37. The dashed line 38 outlines the area of the upper thenar eminence 39. When the hand of a user is positioned on the rear handle 12 of rongeur 10, the cushion 32 engages and fills the entire space of the upper thenar eminence 39. The cushion 32 plus the rest of sleeve 22 fill the entire space of all the thenar eminence 40, outlined by the dashed line 41.

The thenar eminence 40 is the body of muscle on the palm 35 of the human hand 34 just beneath the thumb 37. The thenar eminence 40 often takes the brunt of thumb overuse and repetitive strain. There are at least two reasons for this: First, because the thenar eminence contains the primary muscles of finger-to-thumb gripping, activities or occupations that involve repeated or prolonged use of small instruments or fine tools, such as a rongeur, can be associated with thenar eminence fatigue, pain, and overdevelopment. Secondly, since its three constituent muscles (abductor pollicis brevis, flexor pollicis brevis, and opponens pollicis) are some of the thumb's bulkiest, the thenar eminence also provides the lion's share of palm-to-thumb grip strength when using hand held tools. Two possibilities that cause pain at this fleshy area of the thenar eminence are an inflammation of the flexor tendon to the thumb (flexor tenosynovitis) or a compression of the median nerve at the level of the pronator teres muscle in the forearm. A flexor tenosynovitis of thumb tendons may manifest as pain or locking of the thumb after flexing it.

The horn 27 of a rongeur 10 engages the junction 42 between the thumb 37 and the forefinger 43 when a user grasps the handle 12 of the rongeur 10 (see FIG. 11). Repetitive operation of the handles of the rongeur, as occurs during surgery, produces fatigue, pain, and repetitive motion injuries to the muscles and nerves of the thenar eminence. The sleeve 22 of the present invention, especially with the cushion 32 of the flange 31 around the horn member cover 27, reduces or eliminates fatigue and trauma to the thenar muscles and surrounding nerves, thereby eliminating repetitive motion injuries resulting from operating the rongeur. The sleeve evenly disperses the forces and shock that are the results of operating the instrument. Also, because the sleeve fully engages the space of the thenar eminence, especially at the junction of the thumb and forefinger, the user can more effectively keep a ronguer remarkably steady during surgical use. As a result, the accuracy of surgery with the rongeur is greatly increased when using the sleeve of the present invention.

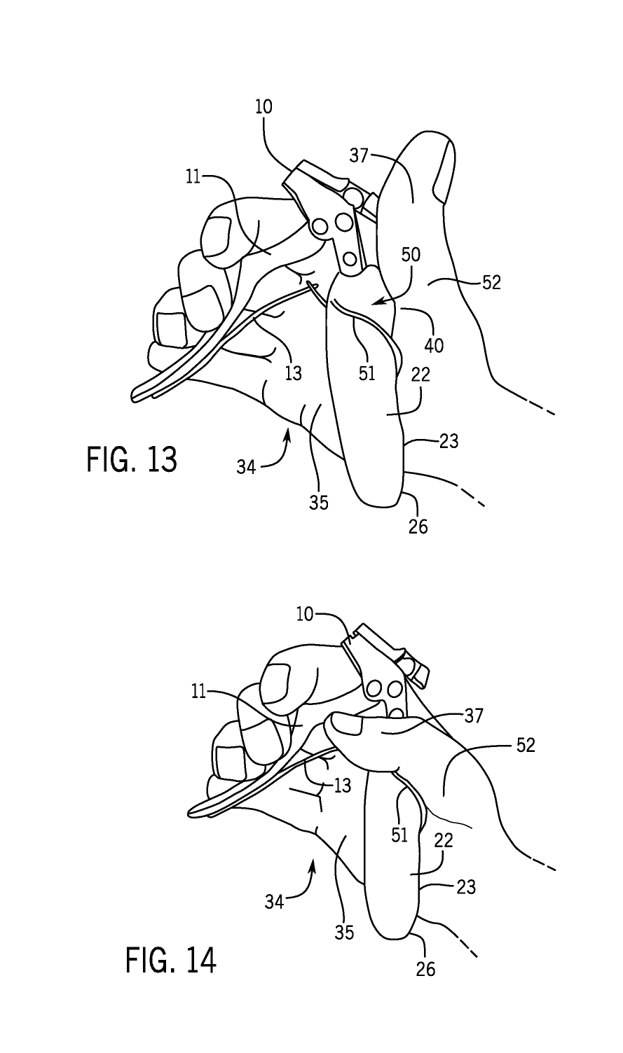

FIGS. 12-14 illustrate another embodiment of the rear sleeve 22. FIG. 12 is a top perspective view of the rear sleeve 22. A projection extends outward from the sleeve 22 at the base of the horn member cover 27, providing a support 50 for the thumb. The thumb support 50 has an outer edge 51. FIG. 13 shows a side perspective view of the rear sleeve 22 positioned in the palmer surface 35 of a hand 34. The inner aspect 52 of the thumb 37 is shown positioned over the thumb support 50. FIG. 14 also shows a side perspective view of the rear sleeve 22 positioned in the palmer surface 35 of a hand 34. The inner aspect 52 of the thumb 37 is shown positioned in the thumb support 50. The thumb support 50 is constructed to accommodate the shape of the inner aspect of the thumb 37 and is made of the same material as the rest of the sleeve 22. The addition of the thumb support 50 to the sleeve 22 gives the user dramatically increased control over the use of the rongeur 10. The sleeve can be constructed to have a right handed or left handed thumb support.

Several embodiments of the handle sleeve of this invention may be constructed and may be used on any device having a handle. The elastomeric material may be formed in different colors, reflective or glowing for ease of identification during a surgery. The handle sleeve may have an indicator strip that will change color after the handle sleeve is released from a sterile environment and may be coated with an antibacterial agent. The elastomeric material may be formulated to decompose after more than one sterile cleaning to ensure only one use or it may be formulated to be reprocessed for multiple uses. The handle sleeve may contain a sensor to determine the number of contractions of the handle (ie. cuts of the rongeur), a sensor which measures the amount of force applied to the handle, a battery for an attached light, camera or other electrical attachment, or a combination thereof. The handle sleeve can be contoured for use on a ring handle, a grooved handle, a hollow handle, or a handle with a horn. Besides being useful on the handles of a rongeur, the handle sleeve can be used on the handles of forceps, mallets, rasps, files, chisels, curettes, saws, bone cutters, lancets, osteotomes, retractors, scissors, surgical spoons, surgical hooks, tweezers, elevators, bone awls, or tamps. The handle sleeve can be contoured for use on any type of rongeur, including rongeurs wherein the surgical handle style is style I ring, style II kerrison, style III love-kerrison, style IV improved love-kerrison, style V ferris-smith kerrison, or style VI ferris-smith kerrison ring.

The foregoing description has been limited to specific embodiments of this invention. It will be apparent, however, that variations and modifications may be made, by those skilled in the art, to the disclosed embodiments of the invention, with the attainment of some or all of its advantages and without departing from the spirit and scope of the present invention.

We claim:

1. An elastomeric sleeve for a handle, comprising,
a) a hollow tube having a first side, a second opposite side, a top end, and a bottom end, wherein said top end is open and said bottom end is closed;
b) said second opposite side has a slit extending from said top end towards said bottom end; and
c) said sleeve is made of compressible material.

2. The elastomeric sleeve of claim 1 further comprising said slit having an opening.

3. The elastomeric sleeve of claim 1 further comprising a cover for a horn member on a handle.

4. The elastomeric sleeve of claim 2 wherein said cover has a flange at a base of said cover, said flange forming a cushion.

5. The elastomeric sleeve of claim 1 further comprising a thumb support.

6. The elastomeric sleeve of claim 5 wherein said thumb support extends from said base of said cover.

7. An elastomeric sleeve for a handle, comprising,
a) a hollow tube having a first side, a second opposite side, a top end, and a bottom end, wherein said top end is open and said bottom end is closed;
b) said second opposite side has a slit extending from said top end towards said bottom end;
c) said sleeve is made of compressible material;
d) said slit having an opening; and
e) a cover for a horn member on a handle.

8. The elastomeric sleeve of claim 7 wherein said cover has a flange at a base of said cover, said flange forming a cushion.

9. The elastomeric sleeve of claim 7 further comprising a thumb support.

10. The elastomeric sleeve of claim 9 wherein said thumb support extends from said base of said cover.

11. An elastomeric sleeve for a handle, comprising,
a) a hollow tube having a first side, a second opposite side, a top end, and a bottom end, wherein said top end is open and said bottom end is closed;
b) said second opposite side has a slit extending from said top end towards said bottom end;
c) said slit having an opening;
d) a cover for a horn member on a handle;
e) said cover having a flange at a base of said cover, said flange forming a cushion; and
f) a thumb support.

12. The elastomeric sleeve of claim 11 wherein said thumb support extends from said base of said cover.
